SEQUENCE LISTING (4 sequences)

SEQ ID NO: 1 - DNA, 13 bp, Homo sapiens
ctgagactgcacc

SEQ ID NO: 2 - DNA, 15 bp, Homo sapiens
gctgagactgcacca

SEQ ID NO: 3 - DNA, 13 bp, Homo sapiens
ctgagattgcacc

SEQ ID NO: 4 - DNA, 15 bp, Homo sapiens
gctgagattgcacca

(19) United States
(12) Patent Application Publication
(10) Pub. No.: US 2006/0057615 A1
(43) Pub. Date: Mar. 16, 2006

(54) Risk prediction for hypertension, elevated plasma triglyceride and metabolic syndrome

(75) Inventors: Wen-Harn Pan, Taipei (TW); Yuh-Shan Jou, Taipei (TW); Jaw-Wen Chen, Taipei (TW); Cathy S. J. Fann, Taipei (TW)

Correspondence Address: FISH & RICHARDSON P.C., PO BOX 1022, MINNEAPOLIS, MN, 55440-1022, US

(73) Assignee: Academia Sinica, Taipei (TW)

(21) Appl. No.: 11/200,650

(22) Filed: Aug. 9, 2005

Related U.S. Application Data
(60) Provisional application No. 60/600,137, filed on Aug. 9, 2004.

(51) Int. Cl. C12Q 1/68

(52) U.S. Cl. 435/6

(57) ABSTRACT

The lipoprotein lipase (LPL) gene is associated with hypertension, elevated plasma triglyceride and metabolic syndrome. A haplotype in the intron 3-intron 4 region (the C/AA/T haplotype), and a haplotype in the intron 5-intron 6 region (the GTAG haplotype), are particularly useful in predicting the risk of an individual for these medical conditions. In addition, other markers that display a linkage disequilibrium with either of these haplotypes can also be used in the same manner. The invention can be applied to Chinese, other Asian populations, Caucasian, African American, and other ethnic groups.

### TECHNICAL FIELD

This invention pertains to risk factors for hypertension, elevated plasma triglyceride and metabolic syndrome, as well as related compositions and uses thereof.

### REFERENCES

1. Mein C A, Caulfield M J, Dobson R J, and Munroe P B. Genetics of essential hypertension. Human Molecular Genetics 2004; 13:R169-75.
2. Harrap S B. Genetic approaches to hypertension. Pages 504-18 in: The textbook of hypertension by John Swales (ed.) Boston, Blackwell Scientific, 1994.
3. Ward R. Familial aggregation and genetic epidemiology of blood pressure. In: Laragh J. H. and Brenner B M, eds. Hypertension: Pathophysiology, Diagnosis, and Management. Vol. 1. New York: Raven Press, 1990:81-100.
4. Hopkins P N, Gerardo H, Ellison C, et al. Coronary artery disease risk in familial combined hyperlipidemia and familial hypertriglyceridemia: a case-control comparison from the National Heart, Lung, and Blood Institute Family Heart Study. Circulation 2003; 108: 519-23.
5. Williams R R, Hunt S C, Hopkins P N, et al. Familial dyslipidemic hypertension: evidence from 58 Utah families for a syndrome present in approximately 12% of patients with essential hypertension. JAMA 1988; 259: 3579-86.
6. Pan W H, Chen Y C, Yu S L, Sun J A, Chang Y S, Chen C J. Correlates and predictive models for blood pressure values in residents of two communities in Taiwan. J Formos Med Assoc 1994; 93:582-91.
7. Shieh S M, Shen M, Fuh M M, Chen Y D, and Reaven G M. Plasma lipid and lipoprotein concentration in Chinese males with coronary artery disease, with and without hypertension. Atherosclerosis 1987; 67: 49-55.
8. Reaven G M, Lithell H, and Landsberg L. Hypertension and associated metabolic abnormalities—the role of insulin resistance and the sympathoandernal system. N Engl J Med 1996; 334:374-81.
9. Grundy S M, Brewer H B, Cleeman J I et al. Definition of metabolic syndrome: Report of the National Heat, Lung, and Blood pressure Institute/American Heart Association Conference on Scientific Issues Related to Definition. Circulation 2004; 109: 433-38.
10. Sprecher D L, Harris B V, Stein Ea et al. Higher triglycerides, lower high-density lipoprotein cholesterol, and higher systolic blood pressure in lipoprotein lipase-deficient heterozygotes: a preliminary report. Circulation 1996; 94:3239-45.
11. Williams R R, Hunt S C, Hopkins P N et al. Evidence for single gene contributions to hypertension and lipid disturbances: definition, genetics, and clinical significance. Clin Genet 1994, 46: 80-87.
12. Mead J R, Irvine S A, and Ramji D P. Lipoprotein lipase: structure function, regulation, and role in disease. J Mol Med 2002; 80: 753-69.
13. Goodarzi M O, Guo X, Taylor K D et al. Lipoprotein lipase is a gene for insulin resistance in Mexican Americans. Diabetes 2004, 53: 214-20.
14. Kastelein J J P, Jukema J W, Zwinderman A H et al. Lipoprotein lipase activity is associated with severity of angina pectoris. Circulation 2000; 102:1629-33.
15. Pan W H, Chen J W, Fann C, Jou Y S, Wu S Y. Linkage analysis with candidate genes: the Taiwan young-onset hypertension genetic study. Hum Genet 2000; 107: 210-5.
16. Wu D A, Bu X, Warden C H et al. Quantitative trait locus mapping of human blood pressure to a genetic region at or near the lipoprotein lipase gene locus on chromosome 8p22. Clin Invest 1996; 97: 2111-8.
17. Hunt S C, Province M A, Atwood L D, et al. No linkage of the lipoprotein lipase locus to hypertension in Caucasians. J Hypertens 1999; 17: 39-43.
18. Clee S M, Loubser O, Collins J, Kastelein J J P, Hayden M R. The LPL S447X cSNP is associated with decreased blood pressure and plasma triglycerides, and reduced risk of coronary artery disease. Clin Genet 2001; 60:293-300.
19. Yang W J, Huang J F, Yao C L, et al. Evidence for linkage and association of the markers near the LPL gene with hypertension in Chinese families. J Med Genet 2003; 40: e57.
20. Ma Y Q, Thomas G N, Critchley Julian A J H, et al. Association of the D8S282 marker near the lipoprotein lipase gene locus with systolic blood pressure in healthy Chinese subjects. Journal of hypertension 2002; 20: 2199-204.
21. Third report of National Cholesterol Education Program (NCEP) expert panel on detection, evaluation, and treatment of high blood cholesterol in adults (Adult Treatment Panel III). Final report. Circulation 2002; 106:3143-421.
22. Pan W H, Flegal K M, Chang H Y, Yeh W T, Yeh C J, Lee W C. Body mass index and obesity-related metabolic disorders in Taiwanese and US whites and blacks: implications for definitions of overweight and obesity for Asians. Am J Clin Nutr 2004; 79: 31-9.
23. Aggarwal R K, Lang T W, Singh L. Isolation of high-molecular-weight DNA from small sample of blood having nucleated erythrocytes, collected, transported and stored at room temperature. Genet Anal Tech Appl 1992; 2: 54-7.
24. Nickerson D A, Taylor S L, Weiss K M, et al. DNA sequence diversity in a 9.7-kb region of the human lipoprotein lipase gene. Nature Genetics 1998; 19: 233-40.
25. Kruglyak L, Daly M J, Reeve-Daly M P, and Lander E S. Parametric and Nonparametric linkage analysis: a unified multipoint approach. Am J Hum Genet 1996; 58:1347-63.
26. Spielman R S, Ewens W J. A sibship test for linkage in the presence of association: the sib transmission/disequilibrium test. Am J Hum Genet 1998; 62: 450-8.
27. Liang K Y, Chiu Y F, Beaty T H, and Wjst M. Multipoint analysis using affected sib pairs: incorporating linkage evidence from unlinked regions. Genet Epidemiol 2001; 21: 105-22.
28. Abecasis GR and Cookson WOC. GOLD-Graphical overview of linkage disequilibrium. Bioinformatics 2000; 16: 182-3.
29. Clayton D, Jones H. Transmission/disequilibrium test for extended marker haplotypes. Am J Hum Genet 1999; 65: 1161-9.
30. Sobel, Lange, Am. J. Hum. Genet. 58, 1323-1337 (1996).
31. Chen J W, Wu S Y, and Pan W H. Clinical characteristics of young-onset hypertension-implications for different genders. Int J Cardiol 96(1), 65-71 (2004).
32. Clark A G; Weiss K M, and Nickerson D A et al. Haplotype structure and population genetic inferences from nucleotide-sequence variation in human lipoprotein lipase. Am J Hum Genet 1998, 63: 595-612.
33. Inskip H and Coggon D. Case-control studies. In: Margetts B M and Nelson M. (eds).

Design Concepts in Nutritional Epidemiology. New York: Oxford University Press, 1997, Vol 1:399-414.
34. WHO expert consultation. Appropriated body-mass index for Asian populations and its implications for policy and intervention strategies. Lancet 2004, 363: 157-163.
35. Merkel M, Eckel R H, Goldberg I J. Lipoprotein lipase: genetics, lipid uptake, and regulation. J Lipid Res 2002, 43: 1997-2006.
36. Tilbeurgh H van, Roussel A, Lalouel J M, Cambillau C. Lipoprotein lipase. Molecular model based on pancreatic lipase X-ray structure: consequences for heparin binding and catalysis. J Biol Chem 1994, 269: 4626-33.
37. Templeton A R, Clark A G, and Weiss K M et al. Recombinational and mutational hotspots within the human lipoprotein lipase gene. Am J Hum Genet 2000, 66: 69-83.
38. Fedorova L and Fedorov A. Introns in gene evolution. Genetica 2003, 118: 123-31.

All of the publications, patent applications, and patents, cited above or elsewhere in this application, are herein incorporated by reference in their entirety to the same extent as if each individual publication, patent application or patent was specifically and individually indicated to be incorporated by reference in its entirety.

### STATE OF THE ART

Hypertension is a major risk factor for cardiovascular diseases and a serious global public health problem. Approximately 1 billion people in the world suffer from this condition (Mein C A, 2004)¹. Evidence from family and epidemiological studies indicates that hypertension arises from a complex interplay between genetic and environmental factors (Harrap S B, Chapter 24 in ref)². Twin studies and segregation analyses have shown that 20%-40% of blood pressure (BP) variation can be attributed to genetic factors (Ward R, 1990)³. Substantial progresses have been made on positional cloning of hypertensive genes for several Mendelian hypertensive traits and on familiar and experimental models of hypertension (Mein C A, 2004)¹. However, efforts to unravel the common genetic variants of this complex trait in humans have not been productive (Mein C A, 2004)¹.

In studies of patients with familial combined hyperlipidemia (FCHL) and familial hypertriglyceridemia (FHTG), hypertension was more frequently observed than in those of normal population (Hopkins P N et al., 2003)⁴. In addition, plasma triglyceride (TG) was significantly associated with blood pressure, and hypertriglyceridemia occurs more often than expected in patients with hypertension (Williams et al, 1988; Pan et al., 1994; Shieh et al., 1987)^{5,6,7}. This clustering phenomenon is recently recognized as part of metabolic syndrome (MS), a condition characterized by a constellation of disorders, including large body mass index (BMI) or waist circumference, elevated plasma TG, low high-density lipoprotein cholesterol (HDL-C), hypertension, and glucose intolerance (Reaven G M, N Eng J Med, 1996)⁸. Numerous lines of evidence suggest that MS plays an important role in the etiology of cardiovascular diseases (Grundy S M et al, 2004)⁹.

Lipoprotein lipase (LPL) is a key enzyme involved in the metabolism of TG-rich particles to release free fatty acid in circulation. Evidence showed that LPL heterozygotes with deficient enzyme activity tend to have higher levels of fasting TG and blood pressure than those with the wild type (Sprecher D L et al, 1996)¹⁰(Williams R R et al., 1994)¹¹. In addition, abnormalities of LPL activity have been associated with insulin resistance, obesity, dyslipidemia with diabetes, chylomicronaemia, atherosclerosis, and Angina Pectoris, etc. (Mead J R et al, 2002)¹² (Goodarzi M O, 2004)¹³ (Kastelein John J P et al, 2000)¹⁴. We, therefore, selected LPL as one of the candidates in our efforts to map hypertensive genes. In our previous report (Pan et al., 2000)¹⁵, using affected sib-pair method, we demonstrated a positive signal linking marker of LPL (D8S1145, p=0.0284) with young-onset hypertension in Taiwan Han Chinese. Linkage between a quantitative trait locus of systolic blood pressure and genetic markers near the LPL gene was also reported in diabetic families of Taiwan (Wu, et al., 1996)¹⁶. Although association of LPL genotype with blood pressure has been controversial in Caucasian studies (Hunt et al, 1999; Clee S M, 2001)^{17,18}, recent independent studies showing positive linkage and association between LPL polymorphism and hypertension in Han Chinese of Mainland China further support the role of LPL in etiology of hypertension (Yang W J, 2003; Ma Y Q, 2002)^{19,20}. It is therefore desirable to develop methods and compositions useful in predicting the risk of, and treating, these medical conditions.

### SUMMARY

Toward a systematic haplotype mapping of LPL to hypertension and related disorders and an understanding of the impact of genetic variations on various facets of such a complex syndrome, we conducted high-resolution mapping of the LPL region for young-onset hypertension. Our results revealed two common LPL haplotypes that are associated with hypertension, elevated triglyceride, and/or the metabolic syndrome (MS).

The first haplotype (the C/AA/T haplotype) involves three single nucleotide polymorphisms (SNPs) in the intron 3-intron 4 region of the LPL gene: rs343 (position 7621708 in NCBI GenBank No. NT_{—}030737), rs252 (position 7622153 in NCBI GenBank No. NT_{—}030737), and rs253 (position 7622338 in NCBI GenBank No. NT_{—}030737). In this risk haplotype, the nucleotides at these three positions are C, AA and T, respectively. It is significantly associated with elevated plasma triglyceride (p=0.005), and with hypertension and elevated plasma TG combined (p<0.0001). The corresponding protective haplotype is C/AAA/C.

The second haplotype (the GTAG haplotype) involves four SNPs in the intron 5-intron 6 region of the LPL gene: AF050163-2500 (position 7624101 in NCBI GenBank No. NT_{—}030737), rs269 (position 7624588 in NCBI GenBank No. NT_{—}030737), rs270 (position 7624597 in NCBI GenBank No. NT_{—}030737) and rs271 (position 7624623 in NCBI GenBank No. NT_{—}030737). In this risk haplotype, the nucleotides at these four positions are G, T, A and G, respectively. The haplotype GTAG was significantly (p<0.0001) associated with combined hypertension and elevated TG. Its corresponding protective haplotype is GTCG.

Furthermore, people who carried both of these risk haplotypes were associated with the highest and significant odds ratio for hypertension (1.5), elevated TG (1.9), hypertension combined with elevated TG (2.3), and metabolic syndrome (1.8).

An additional pair of risk and protective haplotypes were also found in intron 6. The risk haplotype (the ACATT haplotype) comprises A at rs270 (position 7624597 in NCBI GenBank No. NT_{—}030737), C at position 7625325 of NCBI GenBank No. NT_{—}030737, A at position 7625444 in NCBI GenBank No. NT_{—}030737, T at rs281 (position 7625944 in NCBI GenBank No. NT_{—}030737) and T at rs283 (position 7626019 in NCBI GenBank No. NT_{—}030737). The corresponding haplotype comprises C, T, T, A and C at the same positions.

Accordingly, one aspect of the present invention thus provides LPL as a gene for hypertension, elevated triglyceride, MS, and their related diseases and medical conditions. For example, the sequence of the LPL gene or protein can be used to assess the risk of a subject to develop hypertension, elevated triglyceride, MS, or their related diseases or medical conditions. Of particular interest is the use of the LPL haplotypes across the intron 3-intron 4 or intron 5-intron 6 regions, such as the C/AA/T, GTAG, and ACATT haplotypes, as well as their corresponding protective haplotypes. Thus, one aspect of the present invention provides a method for predicting the risk of a subject for hypertension, elevated plasma triglyceride or the metabolic syndrome, comprising investigating the sequence of the lipoprotein lipase (LPL) gene of the subject at the following positions:

(a) positions 7621708, 7622153 and 7622338 of NCBI GenBank No. NT_{—}030737;

(b) positions 7624101, 7624588, 7624597 and 7624623 of NCBI GenBank No. NT_{—}030737; and/or

(c) positions 7624597, 7625325, 7625444, 7625944 and 7626019 of NCBI GenBank No. NT_{—}030737.

In addition to the LPL gene, other markers that display a linkage disequilibrium (LD) with any LPL haplotype that is associated with hypertension, elevated triglyceride, MS, or their related diseases or medical conditions are also so associated. Therefore, these other markers (equivalent genetic markers) can be used to assess the risk for these diseases and medical conditions as well.

The present invention also provides LPL as a target in the development of therapeutic measures for hypertension, elevated triglyceride, MS, and their related diseases and medical conditions. For example, LPL or an LPL variant can be used in an in vitro or in vivo assay to screen for drugs that bind to LPL or the variant, inhibit LPL activity, or restore the activity of the LPL variant. The drugs that have these activities can then be further assayed to determine their use in the prevention or treatment of hypertension, elevated triglyceride, MS, and their related diseases and medical conditions. Particularly useful LPL variants comprise the C/AA/T and/or the GTAG haplotypes.

The present invention also provides LPL nucleic acids that comprise a mutation or polymorphism associated with hypertension, elevated triglyceride, MS, or their related diseases or medical conditions. Proteins or peptides having the mutations or polymorphisms are provided as well. Also provided are antibodies that specifically recognize these variant proteins or peptides, but not the wild-type LPL protein. Vectors and host cells comprising the nucleic acids, as well as anti-sense nucleic acids, are also provided. In particular, primers or probes are provided that can be used to detect LPL mutations or polymorphisms that are associated with hypertension, elevated triglyceride, MS, or their related diseases or medical conditions. Further provided are kits comprising any of the above as well as instructions of use.

### DESCRIPTION OF THE DRAWINGS

FIG. 1 illustrates the relative positions of the components of the LPL gene and SNPs studied in this application. FIG. 1(a) shows a diagram of the LPL gene. The nine exons, E1-E9, are represented by solid vertical bars labeled E1-E9. Bins A-F are shown as filled boxes with the letters A-F below them. The positions of SNPs 1-40 (see Table 3 for details of these SNPs) are shown on the horizontal line above the diagram of the LPL gene. FIG. 1(b) shows the sequences of the common haplotypes in Bins B, D and F. The percentages indicate the percentages of occurrence of each haplotype in the population studied.

FIG. 2 shows the odd ratios of the Bin B and Bin D haplotypes for hypertension, elevated triglyceride, hypertension combined with elevated triglyceride (HTN-TG), and metabolic syndrome. For example, the rightmost columns illustrate the odd ratios of the C/AA/T Bin B haplotype for the metabolic syndrome. For individuals with this haplotype, when Bin D is GTAQ, the odd ratio is 1.8, and when Bin D is GTCC, the odd ratio is 0.9.

FIG. 3 shows the positions of the SNPs in the risk haplotypes and protective haplotypes in the intron 3-intron 6 region of the LPL gene. E4-E7: exons 4-7. Each exon is represented by a solid box labeled as E4, E5, E6 or E7.

### DETAILED DESCRIPTION

The lipoprotein lipase (LPL) gene is associated with hypertension, elevated plasma triglyceride and metabolic syndrome. A haplotype in the intron 3-intron 4 region (the C/AA/T haplotype), and a haplotype in the intron 5-intron 6 region (the GTAG haplotype), are particularly useful in predicting the risk of an individual for these medical conditions. In addition, other markers that display a linkage disequilibrium with either of these haplotypes can also be used in the same manner. The invention can be applied to Chinese, other Asian populations, Caucasian, African American, and other ethnic groups.

Prior to describing the invention in further detail, the terms used in this application are defined as follows unless otherwise indicated.

### DEFINITION

A subject has a “risk” for a medical condition or disease if the probability of the subject to develop the condition or disease is higher than the probability of the general population to develop the condition or disease. The probability of the subject to develop the condition or disease is preferably at least about 1.5 fold, more preferably at least about 2 fold, still more preferably at least about 3, 4, 5, 6, 7, 8 or 9 fold, and most preferably at least about 10 fold as high as the probability of the general population to develop the condition or disease.

A “risk haplotype” is a haplotype that is associated with the risk for a medical condition or disease. Thus, if a subject has the risk haplotype, the probability that this subject develops the condition or disease is higher than a subject who does not carry this haplotype.

A “protective haplotype” is a haplotype that is negatively associated with the risk for a medical condition or disease. Thus, if a subject has the protective haplotype, the probability that this subject develops the condition or disease is lower than a subject who does not carry this haplotype.

A hybridization “probe” is an oligonucleotide that binds in a base-specific manner to a complementary strand of nucleic acid. Such probes include peptide nucleic acids, as described in Nielsen et al., 1991. Probes can be any length suitable for specific hybridization to the target nucleic acid sequence. The most appropriate length of the probe may vary depending upon the hybridization method in which it is being used; for example, particular lengths may be more appropriate for use in microfabricated arrays, while other lengths may be more suitable for use in classical hybridization methods. Such optimizations are known to the skilled artisan. Suitable probes and primers typically range from about 8 nucleotides to about 100 nucleotides in length. For example, probes and primers can be about 8-20, 10-30, 15-40, 50-80, and preferably 12-20 nucleotides in length.

An “equivalent genetic marker” of an SNP or haplotype of interest refers to a genetic marker that is linked to the SNP or haplotype. The useful equivalent genetic markers in the present invention display a linkage disequilibrium with the SNP or haplotype of interest.

“Pharmacogenomics profiling” refers to the determination of genetic factors present in a subject that are associated with diseases or medical conditions, including adverse reactions to drugs. Typically, a panel of genetic factors is determined in pharmacogenomics profiling, and the factors may or may not be associated with the same disease, medical condition, or reaction to drug.

LPL is Associated With Hypertension, Elevated Plasma Triglyceride and Metabolic Syndrome

Hypertension and hypertriglyceridemia are two major components in metabolic syndrome with shared etiology. Therefore, LPL, a major lipid metabolizing gene, has been selected as one of the candidates in our efforts to map hypertension genes in young onset hypertension (YHTN) families.

Recruited were 59 nuclear YHTN families with 25 pairs of affected sibs for the initial linkage study. A confirmatory association study was then carried out with 384 trios from 345 pedigrees. We examined the relations between LPL and hypertension and related phenotypes via the following steps: linkage analysis with microsatellite markers, SNP verification and discovery in the LPL gene, LD analysis, haplotype construction, and family-based association (Examples 1-5).

Microsatellite markers in the LPL gene were linked to YHTN. Transmission disequilibrium tests show that haplotype C/AA/T located in intron 3-4 was significantly associated with elevated plasma triglyceride (TG) (p=0.005), and with hypertension and elevated plasma TG combined (p<0.0001). The haplotype GTAG was significantly (p<0.0001) associated with combined hypertension and elevated TG. Furthermore, people who carried both risk haplotypes were associated with the highest and significant odds ratio for hypertension (1.5), elevated TG (1.9), hypertension combined with elevated TG (2.3), and metabolic syndrome (1.8).

Our results indicate that two common LPL haplotypes are associated with hypertension, elevated TQ, hypertension combined with elevated TG, and MS. The direct involvement of LPL was strongly supported by our initial linkage analysis with microsatellite markers and by the confirmatory haplotype association studies with haplotype SNP markers in the LPL gene. The strongest associations between LPL haplotypes and four MS-related outcomes were detected in patients with combination of risk haplotypes in Bins B (C/AA/T) and D (GTAG), each of which exists in a non-trivial proportions in our population.

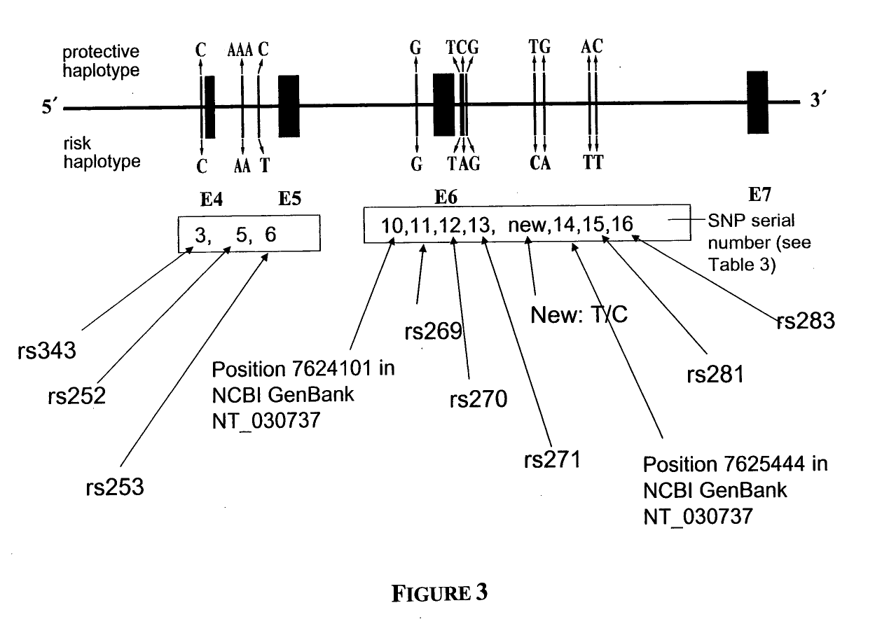

The identified risk haplotypes in both Bin B and Bin D are common variants that make up 21% and 15% of all haplotypes, respectively. The risk haplotype of Bin B was found in 26 of 88 haplotypes discovered by Clark et al (AJHG, 1998)³² in a mixture of Caucasian and African American samples. With regard to Bin D, the risk haplotype we identified was found in 12 of 88 haplotypes of the same study. These two risk haplotypes appear to be common not only in Chinese but also in Caucasian and African American populations. In our young hypertension families, one can pick up around 36% of at-risk individuals in screening with either one of the risk haplotypes. Since the average relative risk of the haplotypes is 1.5 to harvest MS-related phenotypes, the population attributable risk percentage can be estimated to be 15% (Inskip et al., 1997)³³. It indicates that around 15% of the MS-related syndrome may be attributable to the two identified LPL haplotypes in young-onset hypertension families in Taiwan. Therefore, we have identified a common variant for common complex traits: hypertension as well as MS. A third pair of risk and protective haplotypes (ACATT and CTGAC, respectively; FIG. 3) has also been found in intron 6. These haplotypes can be used in the same manner as the C/AA/T and GTAG haplotypes (and their corresponding protective haplotypes).

Almost 100 naturally occurring LPL mutations have been described in various human lipids metabolism disorders, but most of these are rare mutations, including 61 missense mutations, 12 nonsense mutations, 10 frameshift mutations or small insertion/deletions, 3 gross mutations, 8 splicing mutations, and 4 promoter variants (Merkel M et al. J Lipid Res, 2002)³⁵. Among them, more than 58% of these mutations are located in exons 4, 5 and 6 of the LPL gene, where Bins B, C, and D were identified in this study. According to Tilbeurgh H van, et al (1994)³⁶, the LPL protein has two distinct regions: the N-terminal domain (amino acids 1-312, encoded by Exons 1-6), which is responsible for enzymatic catalysis, and the C-terminal domain (amino acids 313-448, encoded by Exons 7-9), which binds to the lipoprotein substrate (Mead J R, 2002)¹². These two domains correspond to the two LD blocks of LPL, which were separated by a 1.9 kb region of recombinational and mutational hotspots located in intron 6, near the exon 6-intron 6 junction (Templeton A R et al, 2000)³⁷. Therefore, LPL variants of Bin B and Bin D, located in introns 3-4 and introns 5-6, respectively, may be related to the catalytic function of LPL. No variations in the promotor region (the proximal 571 bp tested) and exons of the LPL gene were found in the present study except an SNP in exon 8 and Ser447stop in exon 9. Although the protective haplotype of Exons 8-9 had lower-than-expected transmission level to the affected offsprings with elevated triglyceride and hypertension, its degree of association (as determined by transmission disequilibrium tests) was not as strong as that of intron 5-6, and it is not associated with the risk for hypertension or other MS-related phenotypes (data not shown).

It is generally accepted that a polymorphism located in an intron region may be in linkage disequilibrium with a functional mutation either in the coding region or intron-exon junctions of the gene. However, the two risk haplotypes we identified are not in LD with any exonic mutation in the LPL gene. Further work is required to understand how these intronic variants, C/AA/T (crossing exon 4) and GTAG (crossing exon 6), exert their effects. At least six distinct roles of spliceosomal introns have been proposed, including (1) sources of non-coding RNA; (2) carriers of transcription regulatory elements; (3) actors in alternative and trans-splicing; (4) enhancers of meiotic crossing over within coding sequences; (5) substrates for exon shuffling; and (6) signals for mRNA export from the nucleus and nonsense-mediated decay (Fedorova L and Fedorov A, 2003)³⁸.

The significant associations of LPL haplotypes with hypertension and multiple clinical outcomes of MS can be potentially explained. MS is a condition associated with obesity and insulin resistance. Hyperglycemia, hypertension, hypertriglyceridemia and low HDL-cholesterol are the clinical features of MS. It is not altogether clear how these facets of MS relate to one another. Glucotoxicity, lipotoxicity, and obesity induced insulin resistance are among the potential hypotheses. The causal pathway of MS or the sequence of events may very well be multi-directional. Our finding supports that LPL intronic variants may be associated with TG accumulation in blood and tissue, which may in turn contribute to the development of hypertension, hypertriglyceridemia, and/or MS. Literature has documented may other roles of LPL such as: a bridging function to increase cellular uptake of lipoprotein, a monocyte adhesion protein, promoting proliferation of vascular smooth muscle cells, inducing expression of the tumor necrosis factor-alpha gene, stimulating nitric oxide synthetase expression, activating endothelial NAD(P)H oxidase, and reducing the secretion of Apo E (Mead et al, 2002)¹². Many of these mechanisms could also contribute to the development of hypertension.

Thus, the present invention provides a method for predicting the risk of a subject for hypertension, elevated plasma triglyceride or the metabolic syndrome, comprising investigating the sequence of the lipoprotein lipase (LPL) gene of the subject. In particular, the presence of haplotype C/AA/T or haplotype GTAQ or both, is indicative of a risk for these medical conditions. The LPL gene sequence can be investigated using any method established in the art, such as by using an oligonucleotide that specifically hybridizes with the nucleic acid sequence of the haplotype. PCR, PCR-SSCP (single-strand conformation polymorphism), DGGE (denaturant gradient gel electrophoresis), and the ribonuclease A mismatch truncation method are just a few examples of available methods.

It should be noted that in addition to the haplotypes and SNPs described herein, genetic markers that are linked to each of the specific haplotypes and SNPs can be used to predict the risk as well. This is because genetic markers near the haplotypes and SNPs of interest tend to co-segregate, or show a linkage disequilibrium, with the haplotypes and SNPs. Consequently, the presence of these markers (equivalent genetic markers) is indicative of the presence of the haplotypes and SNPs of interest, which, in turn, is indicative of a risk for hypertension, elevated TC, and/or MS.

The equivalent genetic marker can be any marker, including microsatellites and single nucleotide polymorphism markers. Preferably, the useful genetic markers are about 200 kb from the LPL gene or less. More preferably, the markers are about 100 kb, 80 kb, 60 kb, 40 kb, 20 kb, 10 kb, or 5 kb from the LPL gene or less. Linkage disequilibrium can be determined by any method known in the art, including the ones described herein.

The invention also provides nucleic acid fragments (single or double stranded) that encompass each SNP or haplotype described herein. The fragments are usually less than 100, 90, 80, 70, 60, 50, 40, 30, 20 or 15 nucleotides long.

The fragments can be used, for example, as probes. For example, oligonucleotides that specifically bind to the SNP at position 7625325 of NCBI GenBank No. NT_{—}030737 are provided. Oligonucleotides that hybridize to the sequence (or complement thereof) around this position, with C occupying this position, can be used to detect the risk haplotype ACATT. These oligonucleotides thus hybridize to GAGACTGCA, preferably CTGAGACTGCACC, more preferably GCTGAGACTGCACCA (or the complement in each case). On the other hand, oligonucleotides that hybridize to the sequence (or complement thereof) around this position, with T occupying this position, can be used to detect the protective haplotype CTGAC. These oligonucleotides thus hybridize to GAGATTGCA, preferably CTGAGATTGCACC, more preferably GCTGAGATTGCACCA (or the complement in each case). Primers that can be used to detect the SNPs or haplotypes are also provided.

Furthermore, the nucleic acid fragments can be used in various combinations. For example, probes or primers for the three SNPs of haplotype C/AA/T can be included in a kit for detection of that haplotype. Similarly, probes or primers for the four SNPs of haplotype GTAG can be included in a kit. The kit may optionally include additional components, such as:
Hybridization equipments and/or reagents;
PCR equipments and/or reagents;
Primer extension reagents;
Equipments and/or reagents for collecting peripheral blood;
Other oligonucleotide primers or probes;
Instruction of use; and
Container for reagents.

The present invention further provides a method for pharmacogenomic profiling. Thus, a panel of genetic factors is determined for a given individual, and each genetic factor is associated with the predisposition for a disease or medical condition. In accordance with the present invention, haplotype C/AA/T and/or haplotype GTAG are included as markers for hypertension, elevated TG, or MS. The panel may optionally include other genetic factors for the same medical conditions. In addition to markers for hypertension, elevated TG, or MS, the panel may include any other known genetic factors for additional medical conditions or diseases as well.

The following examples are offered to illustrate this invention and are not to be construed in any way as limiting the scope of the present invention. While this invention is particularly shown and described with references to preferred embodiments thereof, it will be understood by those skilled in the art that various changes in form and details may be made therein without departing from the spirit and scope of the invention as defined by the appended claims.

### EXAMPLES

In the examples below, the following abbreviations have the following meanings. Abbreviations not defined have their generally accepted meanings.
° C.=degree Celsius
hr=hour
min=minute
sec or s=second
μM=micromolar
mM=millimolar
M=molar
ml=milliliter
μl=microliter
mg=milligram
μg=microgram
mol=mole
pmol=picomole
ASO=allele specific oligonucleotide
BMI=body mass index
DBP=diastolic blood pressure
HTN=hypertension
LD=linkage disequilibrium
LPL=lipoprotein lipase
MS=metabolic syndrome
NPL=nonparametric linkage
NTP=nucleoside triphosphate
PBS=phosphate buffered saline
PCR=polymerase chain reaction
SBP=systolic blood pressure
SNP=single nucleotide polymorphism
TDT=transmission disequilibrium test
TG=triglyceride
YHTN=young onset hypertension

Materials and Methods

(1) Patients and Families

(A) Initial Linkage and Association Study

Recruited from a hypertension clinic at Taipei Veterans General Hospital were 210 individuals in 59 nuclear families of young-onset hypertension. Among them, there were 25 pairs of affected siblings (Pan, et al., 2000)¹⁵. Their DNA samples were genotyped with 7 microsatellite markers around LPL gene for the initial linkage and association study. Including an additional 9 young-onset hypertension families with elevated TG levels (≧150 mg/dL) recruited later, a total of 267 subjects were used for SNP validation in order to construct LPL haplotypes for confirmatory family-based association study.

(B) Confirmatory Family-Based Association Study

The confirmatory study was carried out in three Hakka communities located in northwest Taiwan. Hakka is Han Chinese who migrated to Taiwan from Canton Province of Mainland China. Young hypertension probands were identified from 4 community hospitals. For every proband identified, his/her family members were invited for blood pressure screening and for a health check-up. A total of 1002 family members of 345 pedigrees were recruited. Among them, 384 trios of young-onset hypertension were used for the confirmatory study. The Human Investigation Committee of the Institute of Biomedical Sciences, Academia Sinica, has approved the protocol of this study.

(2) Definition of Hypertension and Related Phenotypes

Hypertension was defined if any of the following criteria is met: (1) resting systolic blood pressure (SBP) greater than or equal to 140 mmHg, (2) diastolic blood pressure (DBP) greater than or equal to 90 mmHg, (3) currently on antihypertensive medication. Those hypertensive patients with the age of onset under 40 years old were defined as the young-onset cases. In addition, we used two more sub-phenotypes, hypertension combined with elevated TG, and MS for family-based association study. The elevated TG was defined as fasting plasma TG level greater than or equal to 150 mg/dL. A modified NCEP-ATP III definition (Circulation 2002)²¹ was used to define MS. A person was considered to have metabolic syndrome if at least three of the following criteria were met: TG≧150 mg/dL, HDL-C<40 mg/dL for men and <50 mg/dL for women, higher BMI (≧27), high BP (SBP≧135, DBP≧85 mmHg, or on anti-hypertensive medication), and fasting glucose ≧110 mg/dL (or currently on diabetic treatment). The Taiwanese BMI cut-off points for obesity were adopted for this study (Pan et al, AJCN 2004)²².

(3) Genotyping Microsatellite Markers for Initial Linkage and Association Analysis

Genomic DNA of the 59 probands and 151 of their relatives were isolated from collected peripheral lymphocytes by the phenol/chloroform extraction method (Aggarwal et al., 1992)²³. Eight polymorphic microsatellite markers located at 8p22 were genotyped, including D8S1731 (31.73 cM), D8S261 (37.04 cM), D8S1145 (37.04 cM), GZ-14/GZ-15 (GDB:177387 or LPL marker; 39.25 cM), D8S322 (41.55 cM), D8S1116 (42.85 cM), and D8S382 (51.15 cM). The genotyping protocol for microsatellite markers was performed as previously reported (Pan et al, 2000)¹⁵. Fragment analysis was performed using an ABI 377 DNA sequencer and analyzed by GeneScan version 3.0 and genotyper version 3.0. The allele calling was conducted and cross-checked independently by two persons.

(4) Discovery and Validation of Single-Nucleotide Polymorphisms (SNPs) of the LPL Gene

The LPL gene contains 10 exons and spans about 30 kb on the short arm of chromosome 8. In order to validate the identified SNPs in the public database (http://www.ncbi.nlm.nih.gov/snpdatabse) and to discover new SNPs of LPL in Han Chinese, we sequenced 571 bp of the LPL promoter region (41 subjects who were among young-onset hypertension families contributing most to the positive NPL score), exons 1-9 of the LPL coding regions (41 subjects), and selected LPL intron regions containing rich published SNPs (12 subjects—described below) (Nickerson D A et al 1998)²⁴. Secondly, we sequenced intron 1, intron 2, and intron 3 in a 13.8 kb LPL region which contains only a few known SNPS, using DNA from 12 subjects (6 young-onset hypertension patients and 6 age- and sex-matched normotensives).

(5) Genotyping SNPs for LD Map Construction and for Association Studies

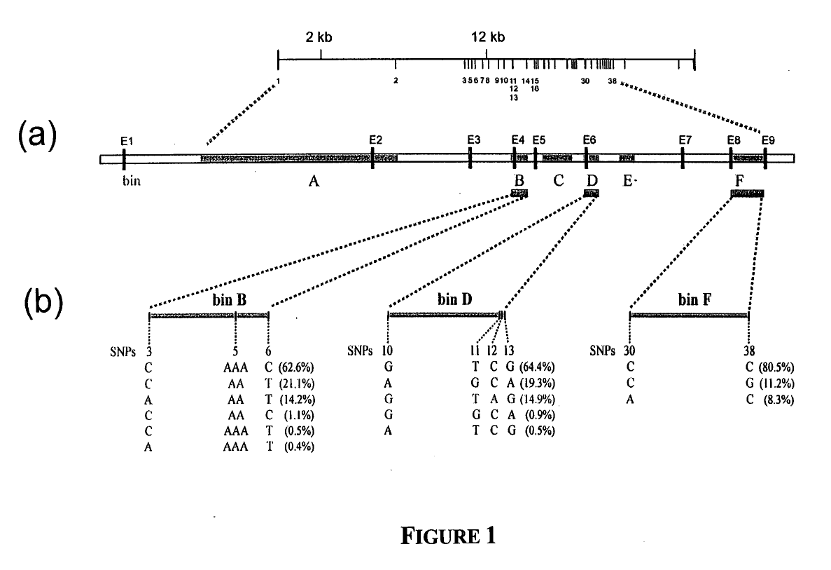

For construction of linkage disequilibrium (LD) map with SNPs within the LPL gene, the newly-identified SNPs and the validated SNPs were typed for 267 Han Chinese by using the MassARRAY SNP genotyping system (SEQUENOM) (Table 3). The polymerase chain reaction was performed with a thermocycler of Perkin Elmer GeneAmp 9700 (Foster City, Calif.) using 10 ng genomic DNA in 20-ul reaction volume. The final concentration of the reagents were 1× AmpliTaqGold PCR buffer (PE/Applied Biosystems, CA, USA), 2.5 mM MgCl₂, 0.2 mM dNTP, 0.2 uM each of primers, and 0.4 U AmpliTaqGold in the reaction mixture. Initial denaturation was performed at 94° C. (for 15 min), followed by 45 cycles of denaturation for 20 s at 94° C., annealing for 30 s at 56° C., and extension for 1 min at 72° C. The final extension was performed at 72° C. for 3 min. The PCR products were extended by extension primers of SNP markers using Homogeneous MassEXTEND spectroPREP™ (SEQUENOM). As indicated in FIG. 1, 9 haplotype SNPs representing Bins B, D, and F were selected for SNP genotyping using 1002 subjects for the confirmatory study. The second SNP in Bins B is polymorphic due to insertion/deletion.

(6) Statistical Analysis

For the initial study, multipoint linkage analysis was performed with 25 pairs of affected sibs from 59 families using the GENEHUNTER program (Kruglyak et al, 1996)²⁵. In addition, information from 42 young hypertensive patients and their parents from 35 families were used to perform the sib transmission disequilibrium test (S-TDT) (Spielman and Ewens, 1998)²⁶. A Bonferroni procedure was carried out to adjust for multiple comparisons²⁶. The Generalized Estimating Equation approach (GEE Version 2.03) (Liang KY et al, 2001)²⁷ was used to compare the phenotypic difference between young-onset hypertension siblings and unaffected siblings using clinical chemistry data including fasting plasma TG.

To construct an LD map for the LPL gene, the 44 identified SNPs with minor allele frequency (MAF) ≧0.05 were tested for linkage disequilibrium by GOLD (Graphical Overview of Linkage Disequilibrium) (Abecasis et al, 2000)²⁸. For the selection and grouping of SNPs, the following parameters were considered: information of LD blocks (D′>0.8), whether the MAF is at or above 0.1, and whether they were significantly associated SNPs and exonic SNPs. The ‘TRANSMIT’ program (version 2.5.2, April 2000) (Clayton and Jones, 1999)²⁹ was used to test for transmission disequilibrium of specific haplotypes to the affected offspring. To solve the problem of dependency between affected trios from families with more than one affected sibs, 10000 bootstraps were carried out in this analysis.

In order to estimate the odds ratio of the SNP haplotypes predicting risk of hypertension and related phenotypes, the SIMWALK program (version 2.86, July 2003) (Sobel, Lange, Am. J Hum. Genet. 1996)³⁰ was used to identify the SNP haplotypes for each individual in the confirmatory study. Then the odds ratios of various SNP haplotypes were estimated by GEE approach using the SAS software (SAS release 8.2).

### Example 1

### Linkage and Association Between Microsatellite Markers and Young-Onset Hypertension

Multipoint analysis showed a positive signal of linkage between young-onset hypertension and the LPL marker located in intron 6 of the LPL gene (GZ-14/GZ-15) with an NPL score of 3 (the number of the independent, affected sib pairs=25). In addition, a significant association was demonstrated by the Spielman's S-TDT with both the LPL marker and D8S322 (Table 1). Allele 1 of the LPL marker (p=1.5×10⁻¹³) and allele 2 of D8S322 (p=3.8×10⁻⁸) were significantly higher in their transmissions to young-onset hypertension offspring than other alleles. Therefore, this analysis indicates that the LPL gene is associated with hypertension.

TABLE 1
Results of Spielman's S-TDT and affected-sib pair analysis
for the initial study

| | | | | TDT | | Com- | | |
|---|---|---|---|---|---|---|---|---|
| | Posi- | | | Transmit | | bined | p-value of | |
| STRP | tion | | | X^{§} | | Z | combined | |
| marker | (cM) | N | Allele | Y^{§} | | score | Z score | NPL^{¶} |
| D8S1145 | 37.04 | 56 | 5 | 28 | 15 | 2.04 | 0.04 | 0.34 |
| | | | 3 | 8 | 17 | 1.33 | 0.18 | |
| LPL | 39.25 | 55 | 1^{†} | 60 | 3 | 7.39 | 1.5 × 10⁻¹³* | 3.02 |
| | | | 3 | 1 | 38 | 6.23 | 4.6 × 10⁻¹⁰* | |
| | | | 2 | 4 | 24 | 3.05 | 0.002 | |
| D8S322 | 41.55 | 49 | 2^{‡} | 38 | 4 | 5.50 | 3.8 × 10⁻⁸* | 1.38 |
| | | | 4 | 11 | 37 | 3.49 | 0.0005 | |
| D8S1116 | 42.85 | 47 | 1 | 4 | 9 | 1.68 | 0.09 | 1.19 |
| D8S382 | 51.15 | 45 | 3 | 8 | 16 | 1.75 | 0.08 | 0.27 |

N: the number of informative trios.
^{†}Allele 1 = 123 bp; Allele 2 = 127 bp; Allele 3 = 131 bp.
^{‡}Allele 2 = 218 bp; Allele 4 = 226 bp.
^{§}X: Number of times that the designated allele was transmitted to affected offspring, when others were not.
^{§}Y: Number of times that the designated allele was not transmitted to affected offspring, when others were.
*P <10⁻⁵ This significance level was set, considering Bonferroni adjustment for multiple markers and alleles.
^{¶}NPL: Non-parametric Lod score for multipoint linkage analysis.

### Example 2

### Clinical Characteristics of the Hypertension Patients and Normotensive Subjects

Clinical findings from the initial linkage study coincide with the above results in that young hypertension patients had significantly higher level of fasting plasma TG (41 mg/dL higher for man, p=0.017, and 33 mg/dL for women, p=0.04) than normotensive controls that were matched for age, sex, residential area and BMI (Chen J W, Int J Cardiol, 2004)³¹. In the confirmatory study, a significant difference in fasting plasma TG was also seen between affected and unaffected siblings (Table 2), as well as most of other MS-related traits including BMI, HDL-C, cholesterol, fasting insulin and glucose.

TABLE 2
Comparing MS-related traits between young-onset hypertension siblings and unaffected
siblings for the confirmatory study

| | Affected siblings | | Unaffected siblings | | | | |
|---|---|---|---|---|---|---|---|
| | mean | | mean | | | | |
| | Male | Female | Male | Female | Difference^{†} | | |
| | (N = 224) | (N = 155) | (N = 161) | (N = 176) | (95% CI) | | p-value* |
| Age (year) | 39.7 ± 6.2 | 40.8 ± 5.5 | 42.1 ± 9.0 | 41.8 ± 9.4 | | | |
| SBP (mmHg) | 139 ± 18 | 137 ± 18 | 124 ± 17 | 118 ± 18 | 19 | (16.6˜21.9) | <0.0001 |
| DBP (mmHg) | 92 ± 13 | 88 ± 12 | 79 ± 12 | 75 ± 12 | 14 | (11.9˜15.6) | <0.0001 |
| BMI (kg/m²) | 27.4 ± 3.9 | 26.0 ± 4.4 | 25.1 ± 3.1 | 23.9 ± 3.8 | 2.2 | (1.7˜2.7) | <0.0001 |
| Waist C (cm) | 91.9 ± 10.0 | 82.7 ± 10.3 | 86.4 ± 8.9 | 76.6 ± 9.8 | 6.3 | (4.9˜7.7) | <0.0001 |
| Triglyceride (mg/dl) | 223 ± 216 | 156 ± 174 | 176 ± 156 | 121 ± 117 | 47 | (25.1˜69.0) | <0.0001 |
| HDL-C (mg/dl) | 43 ± 10 | 52 ± 12 | 47 ± 12 | 55 ± 14 | −2 | (−4.2˜−0.7) | 0.007 |
| LDL-C (mg/dl) | 129 ± 51 | 125 ± 33 | 124 ± 32 | 116 ± 33 | 9 | (2.9˜15.0) | 0.004 |
| Cholesterol (mg/dl) | 211 ± 58 | 202 ± 42 | 200 ± 40 | 191 ± 36 | 14 | (7.05˜21.1) | <0.0001 |
| Insulin (IU/ml) | 18 ± 24 | 16 ± 16 | 14 ± 14 | 12 ± 8 | 3 | (0.9˜4.8) | 0.005 |
| Glucose (mg/dl) | 115 ± 48 | 116 ± 53 | 102 ± 32 | 105 ± 59 | 11 | (3.04˜18.6) | 0.007 |

SBP: systolic blood pressure.
DBP: diastolic blood pressure.
Waist C: waist circumference.
HDL-C: high-density lipoprotein cholesterol.
LDL-C: low-density lipoprotein cholesterol.
The data from 345 families were considered in the mixed model controlling for intra-family correlation, using sex and age as covariates.
^{†}Unaffected siblings as reference group.
*P-value for comparing the log-transformed values.

### Example 3

### The linkage Disequilibrium Map of Single-Nucleotide Polymorphisms Within the LPL Gene and Constructed Haplotypes

To find risk-associated LPL haplotypes for hypertension and other MS-related symptoms, 44 SNPs within the LPL gene (Table 3) were allelotyped with 267 subjects for an LD map. Two major LD blocks were identified from 44 SNPs with an MAF larger than 0.05. We constructed six bins within a 30 kb region of the LPL gene: four in the 5′ terminal LD block (Bins A-D) and two in the 3′ terminal LD block (Bins E-F) (FIG. 1a). Among them, Bins B, D, and F were associated with hypertension.

FIG. 1b shows the most common haplotypes in Bins B, D and F. For Bin B (intron 3-4), three SNPs are shown (SNP3 is rs343, SNP5 is rs252, and SNP6 is rs253). The most common haplotype of Bin B is C/AAA/C (SNP3/SNP5/SNP6). The second most common haplotype, which is risk-associated, is C/AA/T. For Bin D (intron 5-6), the risk-associated haplotype is GTAG (at SNPs 10-13, which are AF050163-2500, rs269, rs270 and rs271, respectively). The risk-associated haplotype of Bin F (exon 8-9) is CG (at SNPs 30 and 38 (rs328)).

TABLE 3
The polymorphisms of LPL gene verified in Taiwan population and their allele frequency

| | | Position in NCBI | | Polymorphisms | | |
|---|---|---|---|---|---|---|
| | Maker | GenBank NT_030737 | | in NT_030737^{‡} | in Taiwanese^{§} | Polymorphisms |
| Marker name | number^{†} | Start | End | (Public name) | (allele frequency) | (allele frequency)^{\|\|} |
| LPL-1322 | | 7606407 | 7606407 | — | A/G (0.96/0.04) | A (1.0) |
| P1-1 | | 7606596 | 7606596 | G/T (rs1470187) | G (1.0) | x |
| P1-2 | | 7606710 | 7606710 | T/C (rs1470186) | T (1.0) | x |
| LPL-53 | | 7607528 | 7607528 | — | C/A (0.83/0.17) | C (1.0) |
| Exon 1 | | 7607873 | 7607960 | | — | |
| | | 7608249 | 7608249 | G/T (rs2898492) | G (1.0) | x |
| I1-2 | | 7608837 | 7608837 | G/T (rs3779787) | G/T (0.71/0.29)* | x |
| I1-4 | | 7610208 | 7610208 | — | A/C (0.96/0.04) | x |
| LPL-55 | 1 | 7610562 | 7610562 | G/T (rs1534649) | G/T (0.71/0.29)* | G/T (0.89/0.11) |
| I1-7-1 | | 7612033 | 7612033 | G/A (rs1031045) | G (1.0) | x |
| LPL-1323 | | 7612451 | 7612451 | — | T/G (0.67/0.33) | x |
| I1-9 | | 7613323 | 7613323 | — | C/T (0.71/0.29)* | x |
| I1-10 | | 7613481 | 7613481 | — | G/A (0.96/0.04) | x |
| I1-11-1 | | 7613779 | 7613779 | — | A/C (0.96/0.04) | A (1.0) |
| I1-11-2 | | 7614014 | 7614014 | C/T (rs3779788) | C/T (0.71/0.29)* | x |
| I1-13-1 | | 7614752 | 7614752 | — | G/A (0.96/0.04) | x |
| I1-13-2 | | 7614917 | 7614917 | — | T/A (0.71/0.29)* | x |
| Exon 2 | | 7616612 | 7616772 | | — | |
| LPL-E2 | | 7616629 | 7616629 | Asp⁹→Asn | | G (1.0) |
| LPL-1868^{¶} | 2 | 7617592 | 7617592 | C/G (rs8176337) | C/G (0.71/0.29) | C/G (0.89/0.11) |
| Exon 3 | | 7620201 | 7620380 | | — | |
| LPL-E3-1 | | 7620215 | 79620215 | Tyr⁶¹→stop | | T (1.0) |
| LPL-E3-2 | | 7620356 | 7620356 | G/A (rs1121923) | | G (1.0) |
| LPL-1870^{¶} | | 7621122 | 7621122 | T/C (rs8176338) | T/C (0.96/0.04) | T/C (0.99/0.01) |
| LPL-1871 | 3 | 7621708 | 7621708 | C/A (rs343) | C/A (0.79/0.21) | C/A (0.87/0.13) |
| | | 7621712 | 7621712 | A/C (rs247) | A (1.0) | x |
| Exon 4 | | 7621742 | 7621853 | | — | |
| LPL-E4 | | 7621747 | 7621747 | G/A (rs248) | | G (1.0) |
| rs249 | 4 | 7621927 | 7621927 | T/C (rs249) | T/C (0.94/0.06) | T/C (0.93/0.07) |
| P4_1_1 | | 7621945 | 7621945 | T—/TG/GT (rs250) | T (1.0) | T (1.0) |
| | | 7622081 | 7622081 | T/C (rs251) | T (1.0) | x |
| P4_1_23 | 5 | 7622153 | 7622153 | A/— (rs252) | AAA/AA (0.75/0.25) | AAA/AA(0.63/0.37) |
| P4_1_4 | 6 | 7622338 | 7622338 | T/C (rs253) | T/C (0.6/0.4) | T/C (0.61/0.39) |
| Exon 5 | | 7622552 | 7622785 | | — | |
| LPL-E5-1 | | 7622617 | 7622617 | G/A (Ala¹⁷⁶→Thr) | | G (1.0) |
| LPL-E5-2 | | 7622654 | 7622654 | G/A (Gly¹⁸⁸→Glu) | | G (1.0) |
| LPL-E5-3 | | 7622672 | 7622672 | T/C (Ile¹⁹⁴→Thr) | | T (1.0) |
| LPL-E5-4 | | 7622703 | 7622703 | C/G (Asp²⁰⁴→Glu) | | C (1.0) |
| P4_2_1 | 7 | 7622818 | 7622818 | C/G (rs254) | C/G (0.625/0.375)^{††} | C/G (0.83/0.17) |
| P4_2_2 | | 7622822 | 7622822 | T/C (rs255) | T/C (0.625/0.375)^{††} | x |
| | | 7622888 | 7622888 | C/T (rs256) | C (1.0) | x |
| | | 7623149 | 7623149 | A/C (rs257) | A (1.0) | x |
| P5_1 | 8 | 7623173 | 7623173 | G/C (rs258) | G/C (0.5/0.5) | G/C (0.60/0.40) |
| | | 7623358 | 7623358 | A/T (rs259) | A (1.0) | x |
| | | 7623420 | 7623420 | C/G (rs260) | C (1.0) | x |
| | | 7623541 | 7623541 | A/G (AF050163-1939) | A (1.0) | x |
| | | 7623563 | 7623563 | A/T (AF050163-1961) | A (1.0) | x |
| P5_2 | 9 | 7623733 | 7623733 | C/T (AF050163-2131) | C/T (0.65/0.35) | C/T (0.73/0.27) |
| P5_3 | 10 | 7624101 | 7624101 | G/A (AF050163-2500) | G (1.0) | G/A (0.82/0.18) |
| rs265 | | 7624190 | 7624190 | C/G (rs265) | x | C (1.0) |
| rs266 | | 7624220 | 7624220 | A/G (rs266) | x | A (1.0) |
| rs267 | | 7624239 | 7624239 | C/T (rs267) | x | x |
| Exon 6 | | 7624273 | 7624515 | | — | |
| LPL-E6-1 | | 7624359 | 7624359 | G/A (Ala²⁶¹→Thr) | | G (1.0) |
| LPL-E6-2 | | 7624450 | 7624450 | A/G (Asn²⁹¹→Ser) | | A (1.0) |
| LPL-1325 | 11 | 7624588 | 7624588 | T/G (rs269) | T/G (0.64/0.36) | T/G (0.83/0.17) |
| | | 7624589 | 7624589 | G/T (rs2075651) | G (1.0) | x |
| LPL-56 | 12 | 7624597 | 7624597 | C/A (rs270) | C/A (0.91/0.09) | C/A (0.82/0.18) |
| LPL-57 | 13 | 7624623 | 7624623 | G/A (rs271) | G/A (0.64/0.36) | G/A (0.82/0.18) |
| | | 7624849 | 7624849 | C/G (rs272) | C (1.0) | x |
| | | 7625154 | 7625154 | C/T (rs275) | C (1.0) | x |
| | | 7625210 | 7625210 | T/C (rs276) | T (1.0) | x |
| LPL-58 | 14 | 7625444 | 7625444 | — | G/A (0.91/0.09) | G/A (0.82/0.18) |
| | | 7625617 | 7625617 | C/G (rs279) | x | x |
| | | 7625803 | 7625803 | G/A (rs280) | G (1.0) | x |
| LPL-59 | 15 | 7625944 | 7625944 | A/T (rs281) | A/T (0.77/0.23) | A/T (0.72/0.28) |
| LPL-60 | | 7625947 | 7625947 | C/G (rs282) | C (1.0) | C (1.0) |
| LPL-61 | | 7625968 | 7625968 | — | T/C (0.96/0.05) | T/C (0.99/0.01) |
| LPL-62 | 16 | 7626019 | 7626019 | C/T (rs283) | C/T (0.82/0.18) | C/T (0.80/0.20) |
| LPL-63 | | 7626027 | 7626027 | T/C (rs284) | T (1.0) | T (1.0) |
| LPL-64 | 17 | 7626110 | 7626110 | C/T (rs285) | x | C/T (0.63/0.37) |
| LPL-65 | | 7626177 | 7626177 | A/T (rs286) | x | A (1.0) |
| LPL-130 | 18 | 7626477 | 7626477 | A/G (rs6586882) | A/G (0.75/0.25) | A/G (0.83/0.17) |
| | | 7626522 | 7626522 | G/A (rs2583659) | G (1.0) | x |
| LPL-131 | 19 | 7626540 | 7626540 | T/C (rs2853630) | T (1.0) | T/C (0.84/0.16) |
| LPL-132 | | 7626759 | 7626759 | G/A (rs2698206) | G (1.0) | G (1.0) |
| LPL-133 | 20 | 7626773 | 7626773 | T/C (rs291) | T/C (0.7/0.3) | T/C (0.92/0.08) |
| | | 7626976 | 7626976 | G/A (rs292) | G (1.0) | x |
| P8_2_1 | | 7627000 | 7627000 | A/— (rs293) | A/— (0.0/1.0) | x |
| P8_2_2 | | 7627046 | 7627046 | T/C (rs294) | T (1.0) | x |
| P8_2_3 | 21 | 7627159 | 7627159 | A/C (rs295) | A/C (0.94/0.06) | A/C (0.85/0.15) |
| | | 7627165 | 7627165 | G/A (rs296) | G (1.0) | x |
| P8_2_4 | | 7627292 | 7627292 | T/C (rs297) | T (1.0) | x |
| Exon 7 | | 7627692 | 7627812 | | — | |
| LPL-E7-1 | | 7627781 | 7627781 | G/A (rs298) | | G (1.0) |
| LPL-E7-2 | | 7627801 | 7627801 | C/T (rs299) | | C (1.0) |
| LPL-E7-3 | | 7627808 | 7627808 | A/G (rs300) | | A (1.0) |
| LPL-66 | 22 | 7627855 | 7627855 | T/C (rs301) | T/C (0.68/0.32) | T/C (0.83/0.17) |
| LPL-67 | | 7627888 | 7627888 | C/T (rs302) | C (1.0) | x |
| LPL-134 | 23 | 7628200 | 7628200 | G/C (rs303) | G/C (0.95/0.05) | G/C (0.91/0.09) |
| LPL-135 | 24 | 7628282 | 7628282 | T/G (rs1963909) | T/G (0.7/0.3) | T/G (0.83/0.17) |
| LPL-136 | 25 | 7628322 | 7628322 | A/G (rs2244603) | A/G (0.7/0.3) | A/G (0.83/0.17) |
| LPL-137 | 26 | 7628397 | 7628397 | — | T/G (0.85/0.15) | T/G (0.93/0.07) |
| LPL-138 | 27 | 7628467 | 7628467 | C/T (rs2853631) | C/T (0.90/0.10) | C/T (0.91/0.09) |
| LPL-150 | | 7628543 | 7628543 | AAAT/— (rs311) | AAAT (1.0) | AAAT (1.0) |
| LPL-68 | 28 | 7628918 | 7628918 | G/C (rs312) | G/C (0.92/0.08) | G/C (0.91/0.09) |
| LPL-69 | | 7628947 | 7628947 | A/G (rs313) | A (1.0) | x |
| LPL-70 | 29 | 7628963 | 7628963 | G/A (rs314) | G/A (0.63/0.38) | G/A (0.83/0.17) |
| LPL-71 | | 7629016 | 7629016 | T/C (rs315) | T (1.0) | x |
| Exon 8 | | 7629333 | 7629515 | | | |
| LPL-E8-1 | 30 | 7629357 | 7629357 | C/A (Thr³⁶¹→Thr) | C/A (0.83/0.17) | C/A (0.91/0.09) |
| LPL-E8-2 | | 7629472 | 7629472 | G/A (Ala⁴⁰⁰→Thr) | | G (1.0) |
| Rs317 | 31 | 7629692 | 7629692 | AG/—or G/T (rs317) | x | G/T (0.91/0.09) |
| LPL-72 | | 7629890 | 7629890 | C/G (rs318) | C (1.0) | C (1.0) |
| LPL-73 | 32 | 7629897 | 7629897 | A/C (rs319) | A/C (0.86/0.14) | A/C (0.80/0.20) |
| LPL-74 | 33 | 7629998 | 7629998 | T/G (rs320) | T/G (0.68/0.32) | T/G (0.83/0.17) |
| LPL-75 | | 7630107 | 7630107 | G/C (rs321) | G (1.0) | G (1.0) |
| LPL-76 | 34 | 7630138 | 7630138 | A/C (rs322) | A/C (0.68/0.32) | A/C (0.83/0.17) |
| | | 7630142 | 7630142 | A/C (rs323) | A (1.0) | x |
| | | 7630143 | 7630143 | A/— (rs324) | A (1.0) | x |
| LPL-77 | 35 | 7630249 | 7630249 | T/C (rs325) | T/C (0.73/0.27) | T/C (0.92/0.08) |
| LPL-78 | 36 | 7630360 | 7630360 | A/G (rs326) | A/G (0.68/0.32) | A/G (0.83/0.17) |
| LPL-79 | 37 | 7630457 | 7630457 | T/G (rs327) | T/G (0.70/0.30) | T/G (0.83/0.17) |
| Exon 9 | | 7630547 | 7630651 | | | |
| LPL-E9 | 38 | 7630645 | 7630645 | C/G (Ser⁴⁴⁷→stop) | C/G (0.95/0.05) | C/G (0.92/0.08) |
| | | | | (rs328) | | |
| | | 7631007 | 7631007 | A/G (rs329) | A (1.0) | x |
| LPL-80 | 39 | 7631317 | 7631317 | G/A (rs330) | G/A (0.92/0.08) | G/A (0.91/0.09) |
| LPL-81 | 40 | 7631326 | 7631326 | G/A (rs331) | G/A (0.92/0.08) | G/A (0.83/0.17) |
| Exon 10 | | 7633742 | 7635690 | | | |
| (3′UTR) | | | | | | |
| P13-1 | | 7634113 | 7634113 | T/C (rs3289) | T/C (0.96/0.04) | T/C (0.996/0.004) |
| | | 7634567 | 7634567 | G/A (rs1059497) | G (1.0) | x |
| LPL-1324 | 41 | 7634569 | 7634569 | A/T (rs3208305) | A/T (0.56/0.44) | A/T (0.84/0.16) |
| P13_3_1 | 42 | 7634595 | 7634595 | — | C/T (0.73/0.27) | C/T (0.9/0.10) |
| P13_3_2 | | 7634884 | 7634884 | C/T (rs1059507) | C/T (0.95/0.05) | x |
| P14_1 | 43 | 7635413 | 7635413 | T/C (rs13702) | T/C (0.73/0.27) | T/C (0.86/0.14) |
| P14_2 | 44 | 7635484 | 7635484 | T/C (rs1059611) | T/C (0.77/0.23) | T/C (0.92/0.08) |
| P14_3 | | 7635588 | 7635588 | C/T (rs15285) | C/T (0.71/0.29) | x |
| P14_4 | | 7635600 | 7635600 | C/A (rs386647) | C/A (0.96/0.04) | x |
| P14_5 | | 7635789 | 7635789 | C/A (rs3916027) | G/A (0.71/0.29) | X |

^{†}The serial numbers of these SNPs were used for the LD and TDT analysis shown in FIGS. 1 and 3 and Table 4.
^{‡}The published SNPs used for verification were obtained from NT_008081 (2001 April version) and NT_008271 (2001 October version).
^{§}The samples used for sequencing were from 12 Taiwanese (6 hypertension patients and 6 normotensive controls). However, for exon SNPs, the samples were from 41 subjects of the young-onset hypertension families. (see Methods)
^{||}The samples used for determining allele frequency were 267subjects in the first study.
*, ^{††}The SNPs with these symbols are in complete LD with one another.
^{¶}LPL-1868 (rs8176337) and LPL-1870 (rs8176338): We submitted these SNPs to NCBI-SNP database.
—: no polymorphism;
x: not genotyped.

### Example 4

### Association of LPL Haplotypes With Young-Onset Hypertension and Other MS-Related Phenotypes in Transmission Disequilibrium Tests

The findings of the initial study with 267 subjects are consistent with those of the confirmatory study for the association between LPL haplotypes and hypertension in transmission disequilibrium tests. In the confirmatory association study with 1505 subjects (expanded from the original 1002) (Table 4), the haplotype C/AA/T of Bin B was significantly higher in its transmission to offsprings with elevated plasma TG (p=0.005), with hypertension and elevated plasma TG combined (p<0.0001), and with metabolic syndrome (p=0.03). In contrast, the C/AAA/C haplotype of block B was significantly lower in its transmission to offsprings with combined phenotypes of hypertension and with elevated TG With regard to Bin D, haplotypes GTCG and GTAG were significantly lower (p<0.0001) and higher (p<0.0001), respectively, in their transmission to offsprings with hypertension and elevated TG combined. Similar positive association with hypertension and with MS were observed for haplotype GTAG in Bin D, but to a less extent. For Bin F, the haplotype CG was inversely associated with the transmission to offspring with elevated level of plasma TG and with hypertension, respectively (p=0.04, p=0.03).

TABLE 4
The TDT-association between hypertension, related phenotypes and SNP haplotypes, using ‘TRANSMIT’
program in the confirmatory SNP study, based on 10000 bootstrap samples

| | | | | | | Bin D (SNP 10, 11, 12, 13)^{‡} | | | | | | | | | |
|---|---|---|---|---|---|---|---|---|---|---|---|---|---|---|---|
| | Bin B (SNP 3, 5, 6)^{‡} | | | | | | Haplo- | | | | Bin F (SNP 30, 38)^{‡} | | | | |
| Phenotype^{†} | N | Haplotype^{§} | Obs | Exp | p value | N | type^{§} | Obs | Exp | p value | N | Haplotype^{§} | Obs | Exp | p value |
| Hypertension | 427 | C/AAA/C | 534 | 537 | 0.665 | 435 | GTCG | 555 | 565 | 0.149 | 431 | CC | 702 | 696 | 0.269 |
| | | C/AA/T | 195 | 188 | 0.176 | | GTAG | 142 | 132 | 0.049 | | CG | 91 | 100 | 0.034 |
| Elevated | 243 | C/AAA/C | 294 | 298 | 0.485 | 251 | GTCG | 312 | 319 | 0.330 | 253 | CC | 415 | 407 | 0.067 |
| triglyceride | | C/AA/T | 118 | 105 | 0.005* | | GTAG | 92 | 75 | 0.062 | | CG | 50 | 58 | 0.037 |
| HTN-ETG | 192 | C/AAA/C | 222 | 235 | 0.007* | 197 | GTCG | 234 | 251 | <0.0001* | 198 | CC | 324 | 321 | 0.446 |
| | | C/AA/T | 105 | 87 | <0.0001* | | GTAG | 80 | 62 | <0.0001* | | CG | 41 | 45 | 0.208 |
| Metabolic | 232 | C/AAA/C | 288 | 289 | 0.875 | 238 | GTCG | 305 | 309 | 0.720 | 236 | CC | 391 | 384 | 0.076 |
| syndrome | | C/AA/T | 113 | 102 | 0.033 | | GTAG | 87 | 73 | 0.022 | | CG | 46 | 52 | 0.094 |

^{†}Phenotype: Elevated triglyceride: fasting plasma triglyceride level ≧150 mg/dl; HTN-ETG: hypertension combined with elevated triglyceride;
^{‡}SNPs number as shown in FIG. 1 and supplementary table 1.
N: The number of affected offspring; Obs: the number of the designate haplotype transmitted to affected offspring; Exp: expected number of transmission.
^{§}Haplotypes with small allele frequency were not shown here.
*It is significant after adjusting for multiple comparisons.

### Example 5

### Odds Ratios of the Risk Haplotypes for Hypertension and Other MS-Related Phenotypes

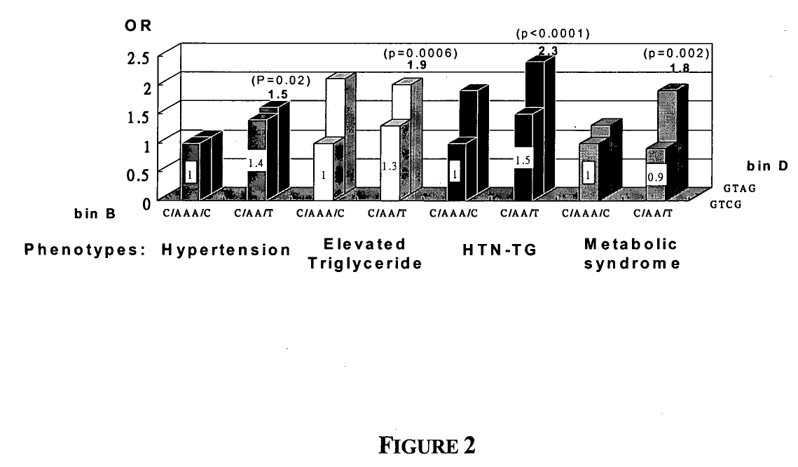

The odd ratio of haplotype C/AA/T of Bin B for estimating risk clinical outcomes was 1.5 (p=0.007), 1.5 (p=0.008), 1.8 (p=0.0001), and 1.3 (p=0.08) for hypertension, elevated TG, hypertension combined with elevated TG, and MS, respectively. The odd ratios of haplotype GTAG of Bin D were 1.5 (p=0.02), 1.7 (p=0.001), 1.9 (p<0.0001), and 1.7 (p=0.002) for the above clinical outcomes, respectively. Haplotypes of Bin F were not associated with any increase of risk to the above clinical outcomes in terms of odds ratio. Furthermore, we classified all people to 4 subgroups by their haplotype status of Bins B and D. People with any one of the risk haplotypes in either Bin B or Bin D tend to have increased odd ratio for most of the above MS-related symptoms including hypertension. Especially, people who carried both risk haplotypes of C/AA/T in Bin B and GTAG in Bin D were associated with the highest and significant odds ratio (1.5 for hypertension, 1.9 for elevated TG, 2.3 for the previous two combined, and 1.8 for MS, respectively) (FIG. 2).

### Example 6

### A new SNP in the LPL Gene

In order to determine whether there are additional SNPs in the LD region that contributed to the disease-association, we sequenced the entire region of intron 4, 5, and 6 of LPL, using samples from 6 homozygotes with risk haplotypes in Bin B and Bin D (C-AA-T-GTAG), and from 6 homozygotes with protective haplotypes (C-AAA-C-GTCG) (FIG. 3). In intron 6, a longer LD block was revealed, spanning from SNP-12 (82 bp away from the exon 6-intron 6 junction) to SNP-16 (1504 bp from the above junction) and in which a new SNP (T/C) was found between SNP-13 and SNP-14. Therefore, haplotype ACATT and haplotype CTGAC of Bin D also can be used to predict risk and protection, respectively.

1. A method for predicting the risk of a subject for hypertension, elevated plasma triglyceride or the metabolic syndrome, comprising investigating the sequence of the lipoprotein lipase (LPL) gene of the subject, wherein a haplotype selected from the group consisting of the following LPL haplotypes is indicative of a risk for hypertension, elevated plasma triglyceride or metabolic syndrome:
(a) haplotype C/AA/T, which comprises C at rs343 (position 7621708 in NCBI GenBank No. NT_{—}030737), AA at rs252 (position 7622153 in NCBI GenBank No. NT_{—}030737), and T at rs253 (position 7622338 in NCBI GenBank No. NT_{—}030737);
(b) haplotype GTAG, which comprises G at AF050163-2500 (position 7624101 in NCBI GenBank No. NT_{—}030737), T at rs269 (position 7624588 in NCBI GenBank No. NT_{—}030737), A at rs270 (position 7624597 in NCBI GenBank No. NT_{—}030737) and G at rs271 (position 7624623 in NCBI GenBank No. NT_{—}030737); and
(c) haplotype ACATT comprises A at rs270, C at position 7625325 of NCBI GenBank No. NT_{—}030737, A at position 7625444 in NCBI GenBank No. NT_{—}030737, T at rs281 (position 7625944 in NCBI GenBank No. NT_{—}030737) and T at rs283 (position 7626019 in NCBI GenBank No. NT_{—}030737).

2. The method of claim 1 wherein the haplotype is C/AA/T.

3. The method of claim 1 wherein the haplotype is GTAG.

4. The method of claim 1 wherein the haplotype comprises both C/AA/T and GTAG.

5. The method of claim 1 wherein the sequence is investigated by using an oligonucleotide that specifically hybridizes with the nucleic acid sequence of the haplotype.

6. The method of claim 1 wherein the sequence is investigated by using DNA prepared from the peripheral blood of the subject.

7. The method of claim 1 wherein the sequence is investigated by using RNA, cells or sera prepared from the peripheral blood of the subject.

8. A method for predicting the risk of a subject for hypertension, elevated plasma triglyceride or the metabolic syndrome, comprising detecting an equivalent genetic marker of a haplotype of the lipoprotein lipase (LPL) gene selected from the group consisting of the following LPL haplotypes:
(a) haplotype C/AA/T, which comprises C at rs343 (position 7621708 in NCBI GenBank No. NT_{—}030737), AA at rs252 (position 7622153 in NCBI GenBank No. NT_{—}030737), and T at rs253 (position 7622338 in NCBI GenBank No. NT_{—}030737);
(b) haplotype GTAG, which comprises G at AF050163-2500 (position 7624101 in NCBI GenBank No. NT_{—}030737), T at rs269 (position 7624588 in NCBI GenBank No. NT_{—}030737), A at rs270 (position 7624597 in NCBI GenBank No. NT_{—}030737) and G at rs271 (position 7624623 in NCBI GenBank No. NT_{—}030737); and
(c) haplotype ACATT comprises A at rs270, C at position 7625325 of NCBI GenBank No. NT_{—}030737, A at position 7625444 in NCBI GenBank No. NT_{—}030737, T at rs281 (position 7625944 in NCBI GenBank No. NT_{—}030737) and T at rs283 (position 7626019 in NCBI GenBank No. NT_{—}030737);
wherein the presence of said equivalent genetic marker is indicative of a risk for hypertension, elevated plasma triglyceride or metabolic syndrome.

9. The method of claim 8 wherein the equivalent genetic marker is detected by using an oligonucleotide that specifically hybridizes with the equivalent genetic marker.

10. The method of claim 1 wherein the equivalent genetic marker is detected by using DNA prepared from the peripheral blood of the subject.

11. A kit for predicting the risk for hypertension, elevated plasma triglyceride or metabolic syndrome, comprising a means for determining the haplotype of the LPL gene.

12. The kit of claim 11 comprising probes or primers for detecting haplotype C/AA/T, which comprises C at rs343 (position 7621708 in NCBI GenBank No. NT_{—}030737), AA at rs252 (position 7622153 in NCBI GenBank No. NT_{—}030737), and T at rs253 (position 7622338 in NCBI GenBank No. NT_{—}030737).

13. The kit of claim 11 comprising probes or primers for detecting haplotype GTAG, which comprises G at AF050163-2500 (position 7624101 in NCBI GenBank No. NT_{—}030737), T at rs269 (position 7624588 in NCBI GenBank No. NT_{—}030737), A at rs270 (position 7624597 in NCBI GenBank No. NT_{—}030737) and G at rs271 (position 7624623 in NCBI GenBank No. NT_{—}030737).

14. The kit of claim 11 comprising probes or primers for detecting haplotype C/AA/T, and probes or primers for detecting haplotype GTAG.

15. The kit of claim 11 comprising oligonucleotides that specifically bind to the sequences of haplotype C/AA/T or haplotype GTAG.

16. The kit of claim 11 further comprising an instruction of use.

17. The kit of claim 11 comprising PCR reagents.
